(19) United States
(12) Patent Application Publication
(10) Pub. No.: US 2016/0228298 A1
(43) Pub. Date: Aug. 11, 2016

(54) COMPRESSION WRAP

(71) Applicant: ICE-SOX, Inc., Los Angeles, CA (US)

(72) Inventors: Daniel Geller, Santa Monica, CA (US); Michelle Geller, Santa Monica, CA (US)

(21) Appl. No.: 15/041,165

(22) Filed: Feb. 11, 2016

Related U.S. Application Data
(60) Provisional application No. 62/114,999, filed on Feb. 11, 2015.

(51) Int. Cl. A61F 13/08; A61F 13/00; A61F 7/02; A61F 13/06

(52) U.S. Cl. CPC A61F 13/08; A61F 13/064; A61F 13/066; A61F 13/068; A61F 13/00038; A61F 7/02; A61F 2013/00093; A61F 2013/00119; A61F 2013/00187; A61F 2013/0028; A61F 2007/0238; A61F 2007/0046

(57) ABSTRACT

A compression wrap in the form of a sock. The sock including a plurality of layers of fabric, the plurality of layers of fabric including an inner layer of fabric and an outer layer of fabric. The sock also including an upper rib band, a middle rib band, a pocket within the inner layer of fabric and the outer layer of fabric and between the upper rib band and the middle rib band, and an opening in the outer layer of the plurality of layers of fabric, the opening providing access to the pocket, the pocket providing support for one or more cold or hot packs.

### CROSS-REFERENCE TO RELATED APPLICATIONS

This application claims the benefit of U.S. Provisional Application No. 62/114,999 filed Feb. 11, 2015. U.S. Provisional Application No. 62/114,999 is incorporated herein by reference in its entirety.

### FIELD OF INVENTION

Embodiments relate to injury relief and more specifically to compression and icing or heating for injuries or post-operatively for individuals having undergone surgical procedures.

### BACKGROUND

The foot is one of the most complex parts of the body, containing 26 bones with 33 joints, 107 ligaments, 19 muscles and multiple tendons holding the structure together to permit the foot to move in a variety of ways. Due to the intricacy of the foot, 75% of Americans experience foot problems at one time or another in their lives. Immediate first aid treatment may prevent complications and promote recovery for injuries such as a sprain, strain, muscle pull, or tear. Post injury or trauma, most doctors will stress the importance of “RICE”, that is, rest, ice, compression and elevation to alleviate some of the symptoms causing discomfort or pain and limit swelling. What is needed is a compression wrap that can provide either icing or heating and allow mobility.

### SUMMARY

In one embodiment, a compression wrap may be in the form of a sock including a plurality of layers of fabric, the plurality of layers of fabric including an inner layer of fabric and an outer layer of fabric, an upper rib band, a middle rib band, a pocket within the inner layer of fabric and the outer layer of fabric and between the upper rib band and the middle rib band, and an opening in the outer layer of the plurality of layers of fabric, the opening providing access to the pocket, the pocket providing support for one or more cold or hot packs. The one or more cold or hot packs may include one or more ice packs or gel packs. The one or more cold or hot packs may provide icing or heating to a person wearing the sock, and may provide the icing or heating to a calf and a shin of a leg of the person simultaneously. The sock may provide compression to a person wearing the sock. The plurality of layers of fabric may include nylon and/or spandex. The one or more cold or hot packs may be inserted into the pocket through the opening. The plurality of layers of fabric may be elastic and the one or more cold or hot packs may be held in place by the elasticity of plurality of layers of fabric. The inner layer of fabric may prevent the one or more cold or hot packs from directly contacting skin. The sock may be capable of being pulled up to below a knee or folded down and pulled over a foot to form additional layers of fabric with compression and permitting icing or heating to a bottom of a foot, a top of the foot, a side of the foot, or an ankle.

In another embodiment, a method may be provided for mobile compression and heat or icing, the method may include wearing a compression sock; the sock may include a plurality of layers of fabric, the plurality of layers of fabric including an inner layer of fabric and an outer layer of fabric, an upper rib band, a middle rib band, a pocket within the inner layer of fabric and the outer layer of fabric and between the upper rib band and the middle rib band, and an opening in the outer layer of the plurality of layers of fabric, the opening providing access to the pocket, the pocket providing support for one or more cold or hot packs; and placing the one or more cold or hot packs into the pocket of the sock through the opening. The method may also include folding down a top portion of the sock including the upper rib band over a foot to provide an additional layer, with compression, to hold the one or more cold or hot packs on a top of a foot, a bottom of the foot, or a side of the foot, or an ankle.

### BRIEF DESCRIPTION OF THE DRAWINGS

The foregoing and other features and advantages of the invention will be apparent from the following, more particular description of various exemplary embodiments, as illustrated in the accompanying drawings wherein like reference numbers generally indicate identical, functionally similar, and/or structurally similar elements. The first digits in the reference number indicate the drawing in which an element first appears.

FIG. 1 depicts an example sock providing a compression wrap being worn by a person up to the knee;

FIG. 2 depicts an embodiment of an example sock providing a compression wrap and heat or cold therapy being worn by a person up to the knee;

FIG. 3 depicts another embodiment of an example sock providing a compression wrap and heat or cold therapy being worn by a person up to the knee;

FIG. 4 depicts an example sock providing a compression wrap and heat or cold therapy being worn by a person over the foot;

FIG. 5 depicts an embodiment of an example sock providing a compression wrap and heat or cold therapy being worn by a person over the foot;

FIG. 6 depicts another embodiment of an example sock providing a compression wrap and heat or cold therapy being worn by a person over the foot;

FIG. 7 depicts a side of an example sock showing an upper rib band, middle rib band, and an opening;

FIG. 8 depicts another side of an example sock showing upper rib band and middle rib band;

FIG. 9 depicts a close up of an example upper rib band;

FIG. 10 depicts an example middle rib band;

FIG. 11 depicts a close up of an example middle rib band;

FIG. 12 depicts an example toe end of example sock;

FIG. 13 depicts an example opening; and

FIG. 14 depicts another depiction of an example opening.

### DESCRIPTION OF THE EMBODIMENTS

Exemplary embodiments are discussed in detail below. While specific exemplary embodiments are discussed, it should be understood that this is done for illustration purposes only. In describing and illustrating the exemplary embodiments, specific terminology is employed for the sake of clarity. However, the embodiments are not intended to be limited to the specific terminology so selected. A person skilled in the relevant art will recognize that other components and configurations may be used without parting from the spirit and scope of the embodiments. It is to be understood that each specific element includes all technical equivalents that operate in a similar manner to accomplish a similar purpose. The examples and embodiments described herein are non-limiting examples.

All publications and references cited herein are hereby incorporated by reference in their entirety.

As used herein, the term “a” refers to one or more. The terms “including,” “for example,” “such as,” “e.g.,” “may be” and the like, are meant to include, but not be limited to, the listed examples. The term gel pack (e.g., ice pack, cold pack, hot pack, etc.) may include material that may provide either cooling (e.g., icing) or heat, gel pack may include other material besides gel such as water or refrigerant or material designed to heat up or cool down a body part. The gel pack may be reusable or disposable and may be “heated” (e.g., via microwave or boiling water) to provide warmth in the event a person is instructed to apply heat the area, or “cooled” (e.g., via refrigerator or freezer) in the event the person is instructed to ice the area.

Rest, ice, compression, and elevation (“RICE”) is often recommended for physical injuries and post-operatively for patients having undergone surgical procedures. In an embodiment of the invention described herein, techniques may be provided to supply compression and cooling or heating, to injuries caused, for example, by day-to-day activities, post-operatively for patients having undergone surgical procedures, or to post workouts to alleviate the “wear and tear” caused by endurance sports. As described below, a sock may be worn that may provide compression and cooling or hearing, while allowing the wearer to have mobility and to elevate.

In one embodiment, a compression wrap may be in the form of a sock (e.g., ICE-SOX). In one embodiment, the sock may be made out of, for example, 86% nylon and 14% spandex to provide elasticity for compression to the wearer. Other embodiments may have varying degrees of nylon and spandex, or other fabrics, to provide elasticity for compression to the wearer. Some embodiments may be made out of synthetic materials, natural materials, or a combination of both. The redesigned sock (e.g., ICE-SOX) may cater to both the athletic community and those individuals suffering from day to day foot and/or ankle injuries, individuals having undergone surgical procedures, or in need of foot or leg compression. The sock (e.g., ICE-SOX) may provide a person with compression from below the knee to the toes of the foot, while also providing the ability to ice or heat the shin, calf, ankle, and/or entire foot, while also providing the person with the ability to elevate the leg or foot or to be mobile. The sock (e.g., ICE-SOX) may be used with one or more reusable packs or pads such as an ice pack or gel pack to provide cooling to an injured area of, for example, the lower leg, foot, or ankle.

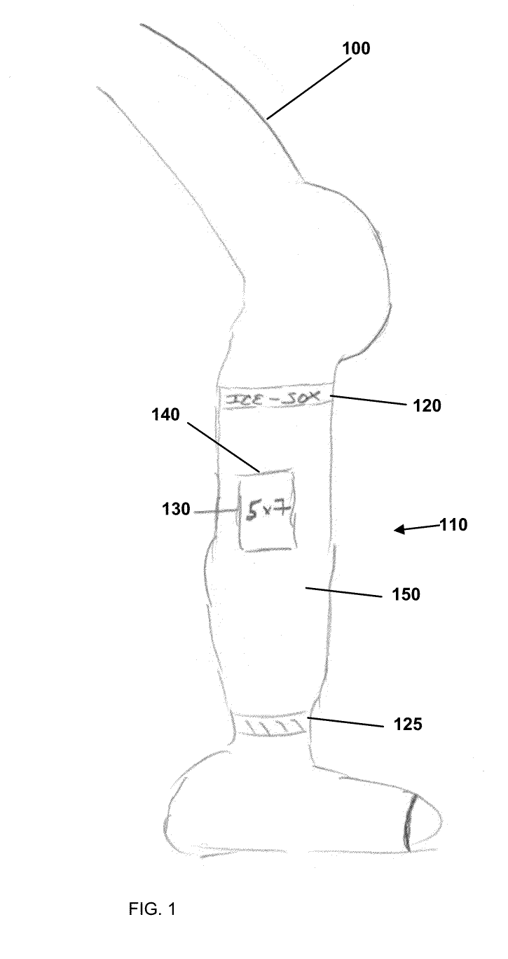

FIG. 1 depicts example sock 110 (e.g., ICE-SOX) providing a compression wrap being worn by wearer 100 (e.g., a person) up to the bottom of the knee of person 100. Sock 110 may include one or more rib bands 120 and 125 in sock 110 providing additional compression. Rib band 120 may be placed at the top of sock 110. Rib band 125 may be placed at a location in sock 110 to match with an ankle of person 100, for example. Rib bands may contain the same material as the rest of sock 110, or may contain different material. Rib bands 120 and 125 may be of different construction and knitted differently from the rest of the sock 110. Rib bands 120 and 125 may provide even greater elasticity than the rest of sock 110. In one embodiment, rib band 120 may be, for example, 1×1 inches and ½ inch wide; and rib band 125 may be, for example, 1×1 inches and 1 inch wide. Other embodiments may have different sizes of rib bands.

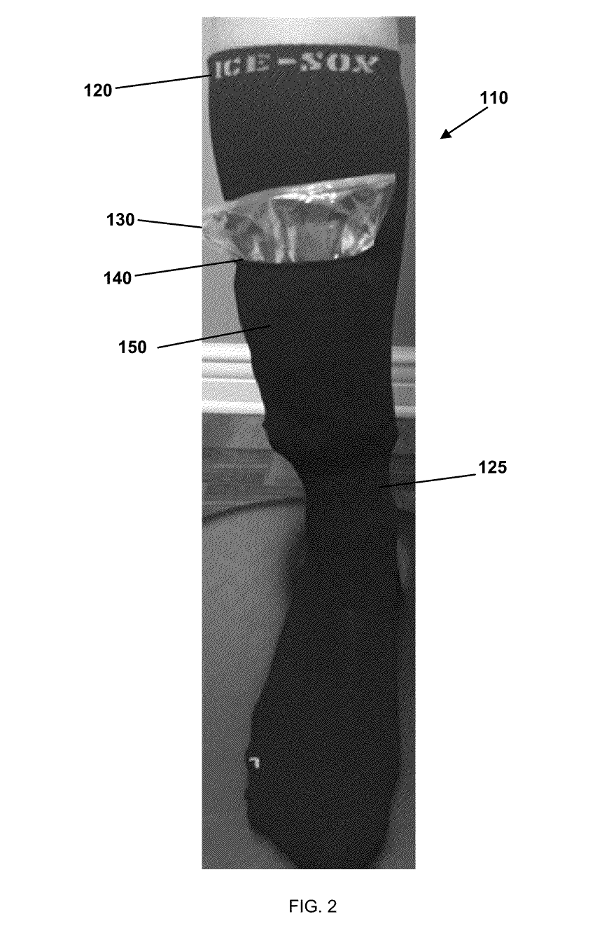

Sock 110 may include a plurality of elastic layers providing compression pressure to the wearer 100 of the sock. Sock 110 may include an inner layer and an outer layer of the plurality of elastic layers. The plurality of layers may be made out of a fabric. An opening 140 or incision in the outer layer may provide access to a pocket 150 between the inner layer and the outer layer. In one embodiment, the opening 140 or incision in the outer layer providing access to pocket 150 may be horizontal, for example as depicted in FIG. 2, or, in another embodiment, opening 140 may be vertical. Opening 140 may be located on the front of sock 110, as depicted in FIG. 2, or the back or side of sock 110.

Pocket 150 may provide support for gel pack 130 (or multiple gel packs) to provide icing or heating to wearer 100. When sock 110 is worn up to the knee, icing or heating may be provided to the shin, calves, or both simultaneously through, for example, the insertion of two or more gel packs 130 into pocket 150 via opening 140. The inner layer of fabric may provide a shield for the skin of wearer 100 to avoid direct contact with the gel pack. Additionally, rib bands 120 and 125, along with the compression component of sock 110, may keep gel pack 130 in the desired location.

FIG. 2 depicts another example of sock 110 (e.g., ICE-SOX) providing a compression wrap being worn by wearer 100 (e.g., a person) up to the bottom of the knee of person 100. In FIG. 2, gel pack 130 is being inserted through opening 140 into pocket 150. Rib bands 120 and 125, along with the compression component of sock 110, may keep gel pack 130 in the desired location.

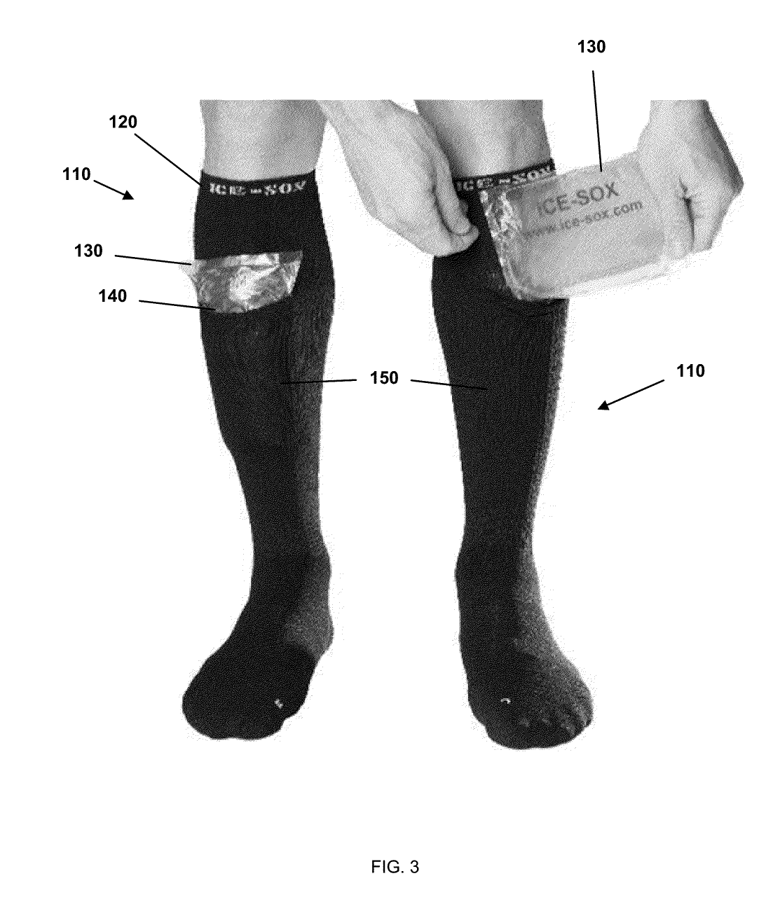

FIG. 3 depicts an example of two socks 110 (e.g., ICE-SOX) providing compression being worn by wearer 100 (e.g., a person) on both legs up to the bottom of the knees of person 100. One or more gel packs 130 may be placed in an opening on, for example, the front of sock 110, although the opening may be on the back of sock 110, for example, and can be moved around the entire leg, shin or calf. Opening 140 may contain additional elasticity, for example, to keep the opening 140 closed and to prevent ice pack 130 from accidentally leaving pocket 150. In some embodiments a fastener, for example, buttons or VELCRO, may be used with opening 140 keep gel pack 130 from accidentally leaving pocket 150. Opening 140 may be kneaded tighter with more ends of yarn, for example, to create additional elasticity. Sock 110 may have multiple layers allowing for gel packs 130 to be inserted into the opening and nestled between the layers preventing direct contact with the skin. One or more gel packs 130 may be moved to desired locations on the leg. The compression and tightness of sock 110 prevents gel pack 130 from inadvertent movement when person 100 is in motion, for example. Additionally, rib bands 120 and 125 prevent further movement of gel pack 130. Person 100 may be mobile while wearing sock 110. For example, person 100 may wear shoes with sock 110 and can be, for example, walking around while compression and hot or cold is being applied. FIGS. 1-3, for example, show sample placement of gel pack 130. In one example, gel pack 130 may be 5 inches by 7 inches in size or smaller or larger. In one embodiment, sock 110 may be provided in men's and women's sizes. In other embodiments, gel pack 130 may be smaller or larger. The men's size may be, for example, 1.5 inches from toe to heel and 11 inches from the ankle to the knee. The women's size may be, for example, 6 inches from toe to heel and 9.5 inches from ankle to knee. In an embodiment, the men's and women's sizes may, for example, “stretch to fit” and may be different sizes from the toe to heel and ankle to knee.

The size of pocket 150 may span the entire circumference of the leg from band 120 to band 125, for example. Gel pack 130 may be placed any location within pocket and held in place, for example, through the compression feature of sock 110 and rib bands 120 and 125.

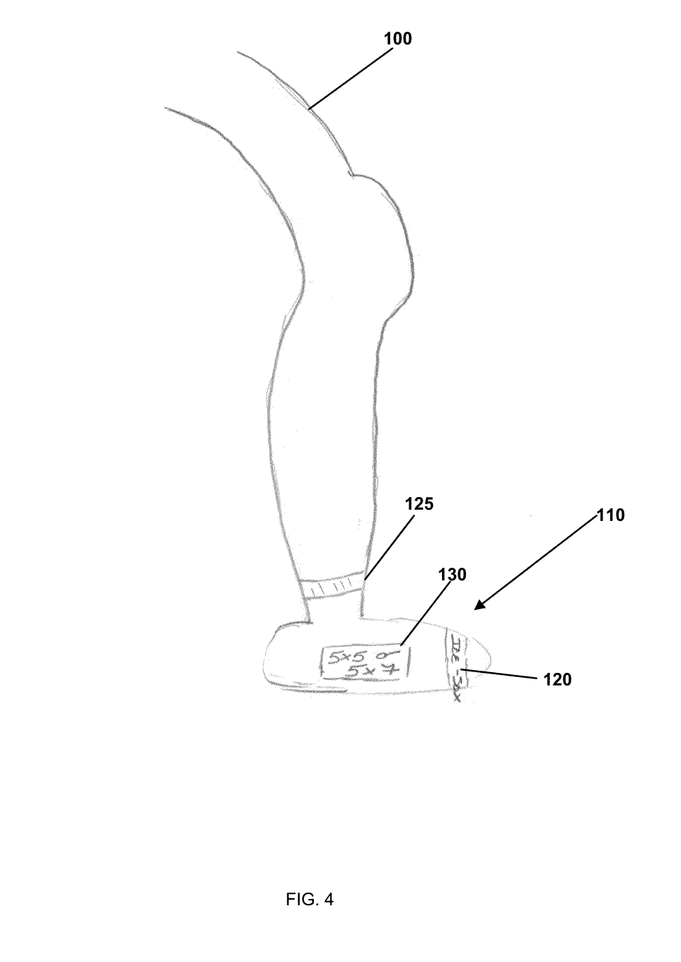

FIG. 4 depicts example sock 110 (e.g., ICE-SOX) providing a compression wrap being worn by person 100 over the foot of person 100. In the embodiment shown in FIG. 4, sock 110 may be folded down and brought over the foot of person 100. Rib band 120 (near the knee in FIG. 1) may be placed near the toes of person 100. One or more gel packs 130 may be placed in an opening below the toes and gel packs 130 may be moved around the entire foot (top, bottom, sides, back) and ankle. The compression, along with the rib band 125 around the ankle and rib band 120 (now near the toes) holds gel packs 130 in place. The construction of sock 110 (e.g., ICE-SOX) may permit person 100 to either be elevating a foot or to be walking around while icing or heating. However, person 100 should not be mobile if they are icing or heating the bottom of the foot as to not apply pressure to gel pack 130, for example. In this example, gel pack 130 may be, for example, 5 inches by 5 inches or 5 inches by 7 inches, although gel packs on other embodiments may be smaller or larger. Additionally, the fabric layer of the sock covering the foot may provide a shield to avoid direct contact of the gel packs 130 with the skin.

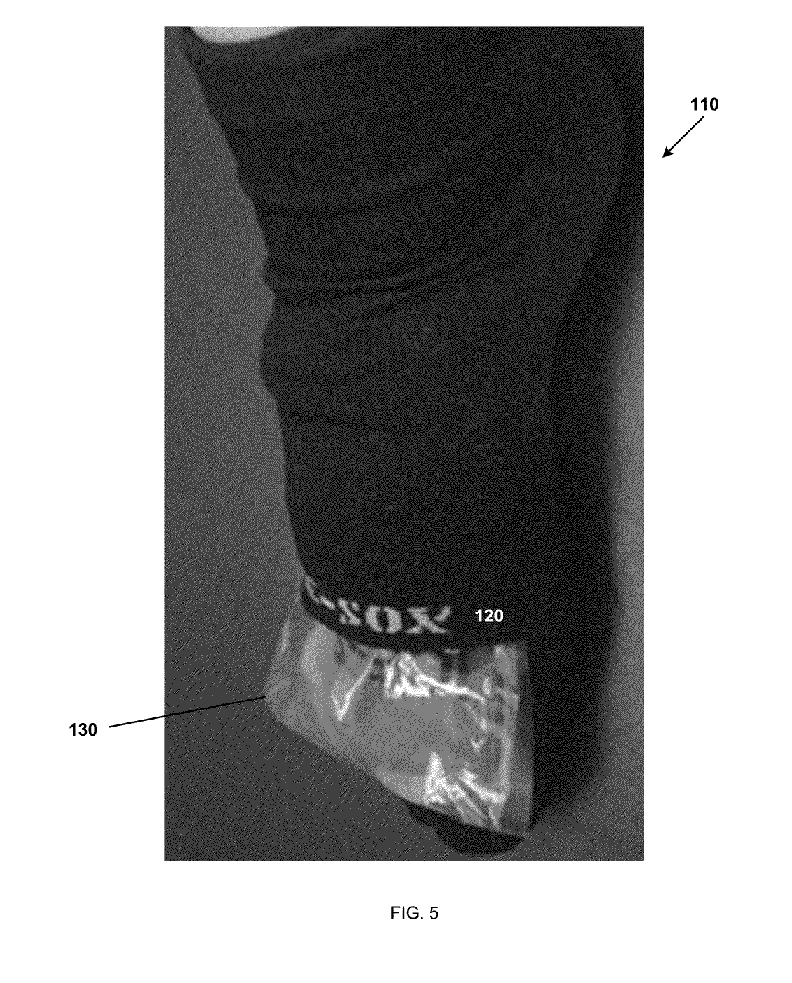

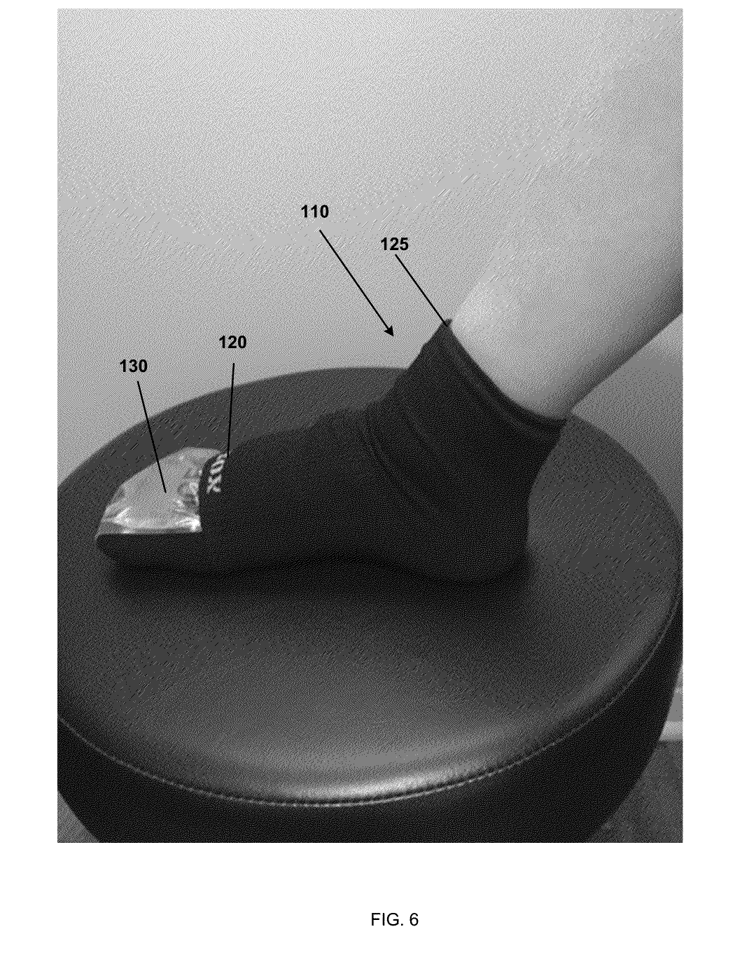

FIGS. 5 and 6 depict sock 110 pulled onto the foot and gel pack 130 inserted. The upper layer of the sock that previously was pulled up below the knee may be folded down and pulled over the foot of person 100 to provide an additional layer of material (e.g., fabric), with compression, to hold one or more gel packs on the top, bottom or side of the foot and ankle, permitting icing (or heating) capabilities to those areas. The rib bands at 120 and 125 of sock 110, for example, keep the gel packs in the desired locations on the foot and ankle. The fabric layer of the sock covering the foot may provide a shield to avoid direct contact of the gel packs 130 with the skin.

Sock 110 whether pulled up to below the knee or folded down around the foot may hold one or more gel packs 130. For example, the size of the pocket 150 may fit two gel packs 130, thus permitting the shin and calves to be iced or heated simultaneously, and same with the foot (top and bottom).

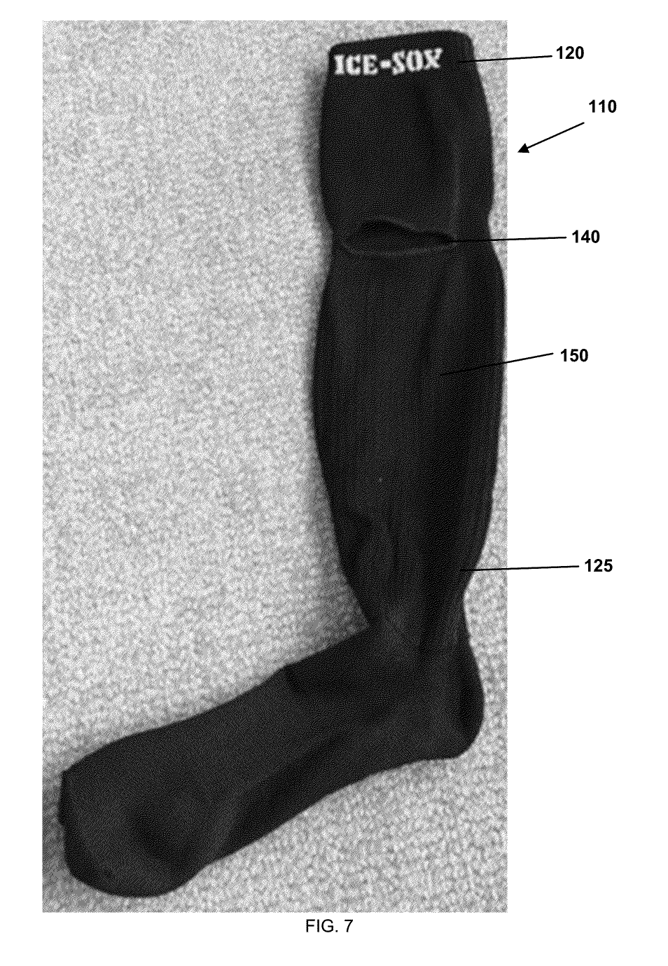

FIG. 7 depicts a side of sock 110 with upper rib band 120, middle rib band 125, and opening 140. Sock 110 may have a double layer (inner layer and outer layer) with an incision or opening 140 (that may be, for example, on the front or back of sock 110, and may be horizontal or vertical) in the outer layer to provide access to pocket 150 to provide support for one or more gel packs 130 to provide icing (or heating) capabilities, when worn up to the knee, to either the shin or calves, or both simultaneously (through the insertion of two gel packs 130 into pocket 150). The size of pocket 150 may span, for example, from band 120 to band 125, and may go around the entire circumference of the leg, thus permitting icing (or heating) to the entire area of the leg between band 120 and 125. The inner layer of pocket 150 may provide a shield for the skin from direct contact with the gel pack(s) 130.

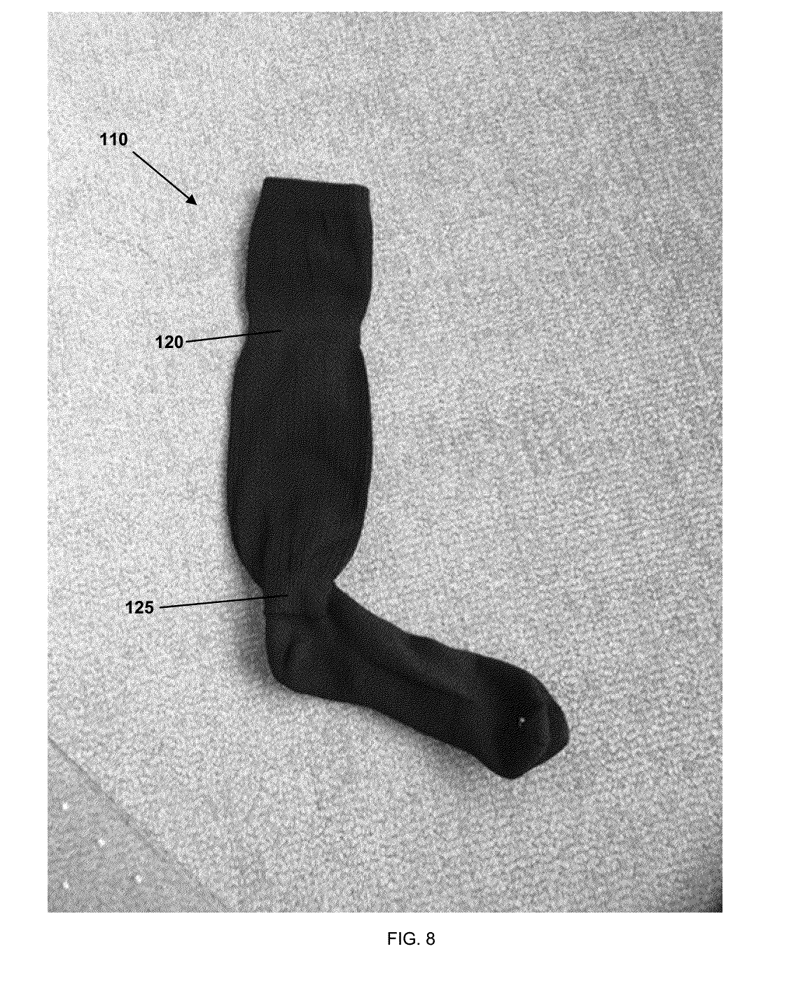

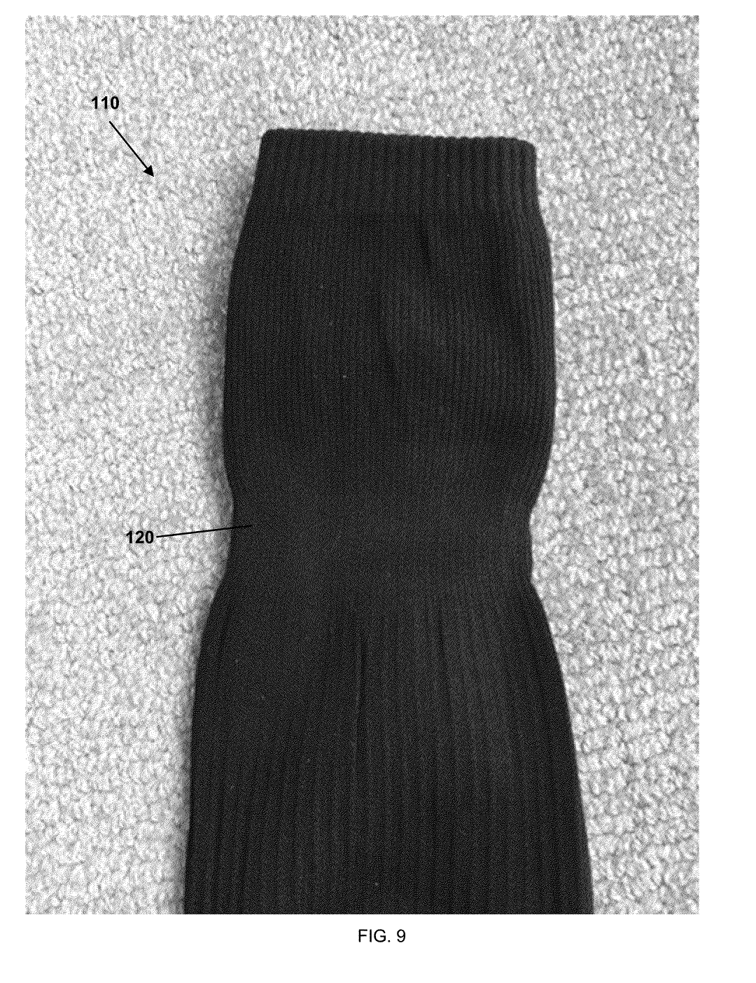

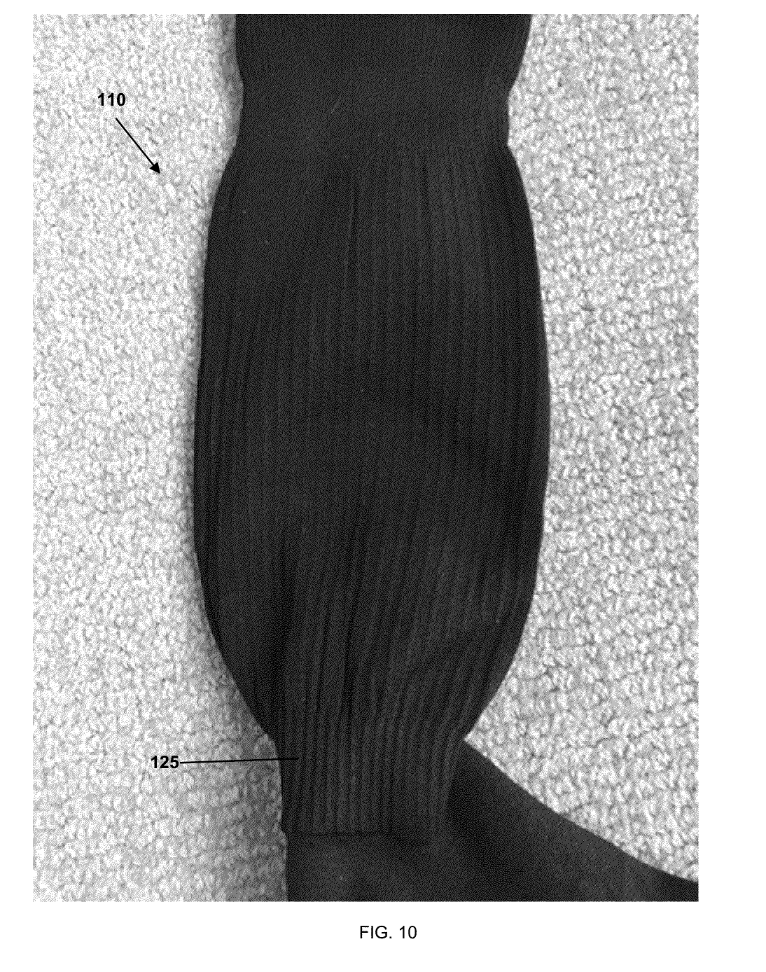

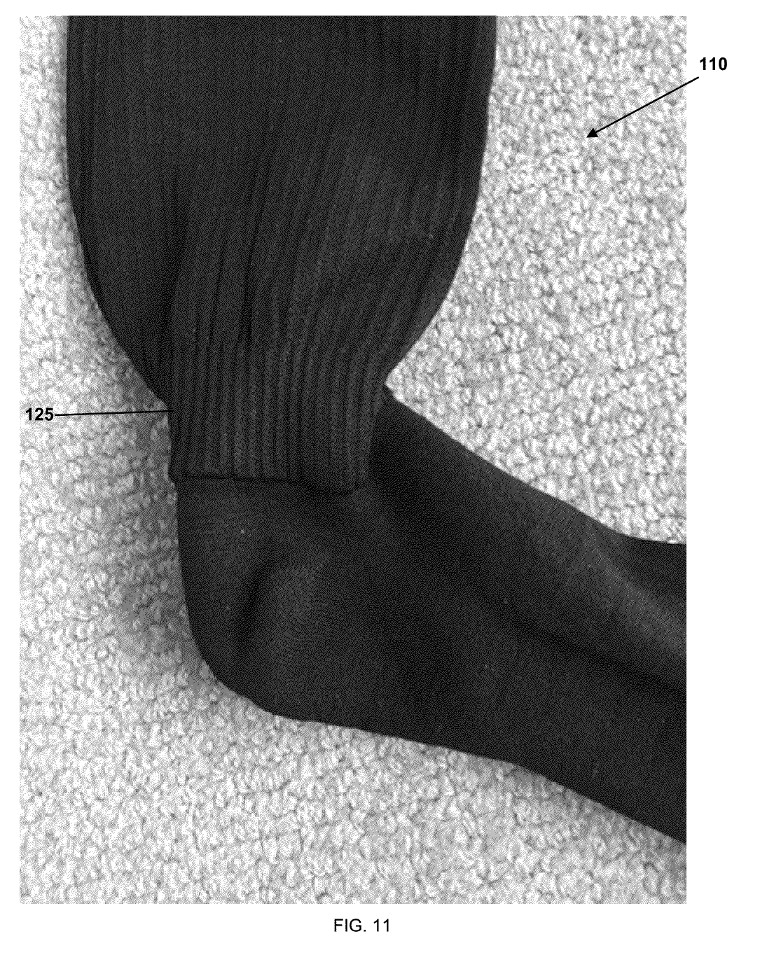

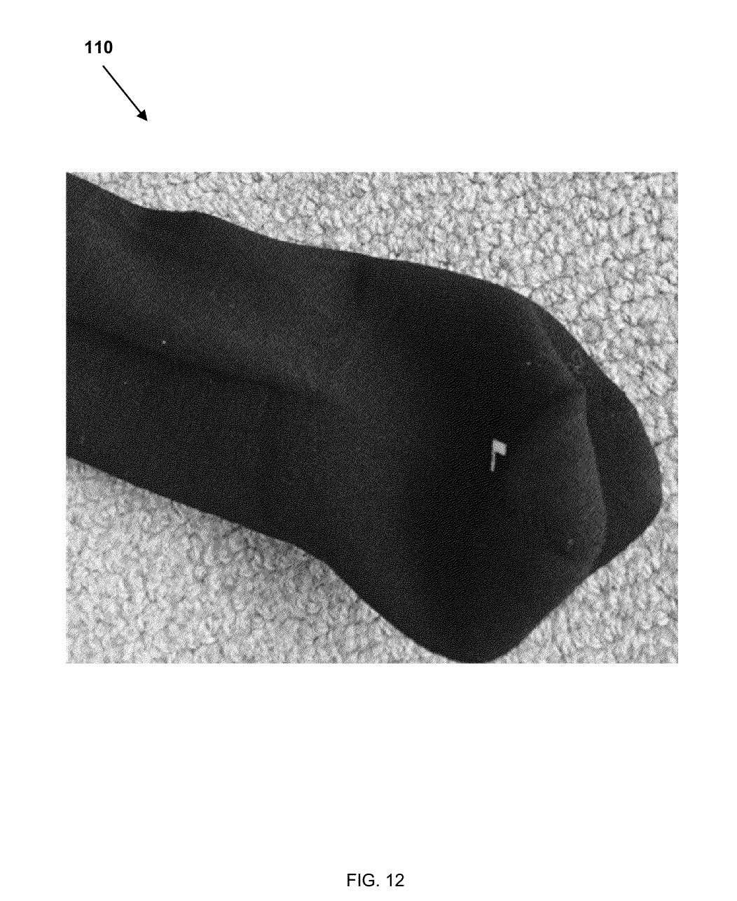

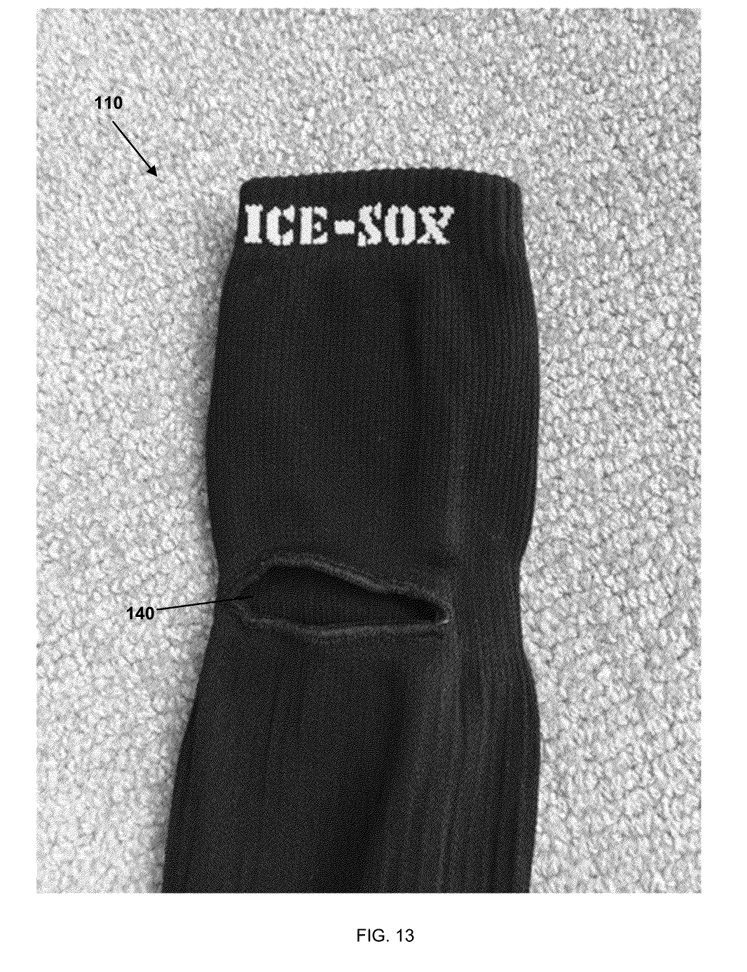

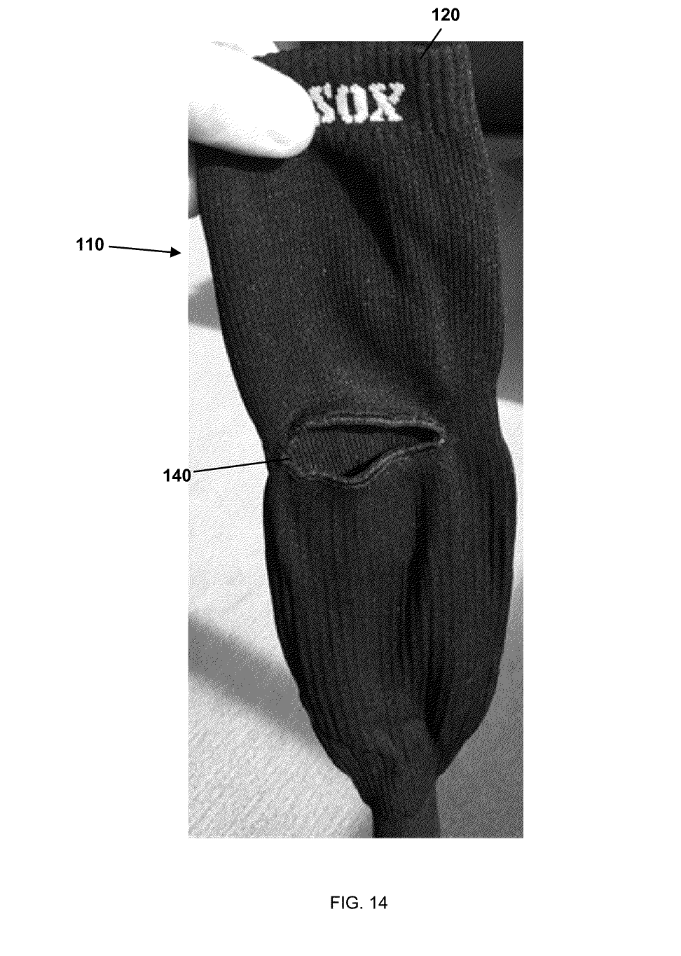

FIG. 8 shows another side of sock 110 with upper rib band 120 and middle rib band 125. FIG. 9 shows a close up of upper rib band 120. FIG. 10 shows middle rib band 125. FIG. 11 shows a close up of middle rib band 125. FIG. 12 shows the toe end of sock 110 with an anatomically accurate design for the left and right feet. FIG. 13 shows opening 140. FIG. 14 shows another depiction of opening 140.

The sock 110 described herein (e.g., ICE-SOX) and shown in FIGS. 1-14 may have multiple advantages over conventional methods of icing the calf, shin, foot & ankle, which often include items like ace bandages, bags of frozen peas, plastic wrap, and towels. As previously noted, “RICE,” or rest, ice, compression and elevation, are important factors in aiding recovery from an injury, whether due to over-use, a surgical procedure, or due to day-to-day activities, and helps prevent over-use injuries caused by endurance sports. Sock 110 may provide at least the following features.

Sock 110 provides the “I” in “RICE,” and permits person 100 to ice (or heat) either the entire calf, shin, foot & ankle. Ice or cooling, for example, is a very important component of recovery, and reduces swelling, fatigue and muscle soreness. In previous techniques, certain areas of the foot, for example, the Achilles tendon or side, were unable to be iced without having the gel pack shift or fall off the injured area. Sock 110 (e.g., ICE-SOX) may allow person 100 to reach these areas, place the gel pack 130 on the injured area, and have the gel pack remain in place while person 100 is mobile or elevating the foot.

Sock 110 may provide the “C” in “RICE.” Compression is a very important component of recovery as it helps control swelling and inflammation around an injured area. Studies have shown applying compression while icing decreases recovery time due to increased oxygenation. Sock 110 (e.g., ICE-SOX) may allow person 100 to easily and functionally, ice (or heat) and compress at the same time.

Sock 110 may provide person 100 to elevate (“E”), while icing (or heating) and compressing the calf, shin, foot and/or ankle of person 100. Prior to sock 110 (e.g., ICE-SOX) icing, compressing, and elevating was not easily accomplished. For example, one component would be missing, or the ice would continually shift or fall off. The compression component of sock 110 (e.g., ICE-SOX), along with rib bands 120 and 125, for example, keeps gel pack 130 in the desired location while mobile or elevating, thus alleviating the need for plastic wrap, ace bandages, towels, etc.

The combination of materials (e.g., 86% nylon and 14% spandex, or other varying percentages and fabrics) used to manufacture Sock 110 (e.g., ICE-SOX), shields the skin from direct hot or cold contact, therefore eliminating the need for towels, shirts or other methods of blocking direct contact with cold on the skin, and keeps gel pack 130 on the injured area alleviating the need for plastic wrap or ace bandages, for example. The combination of material used also does not readily absorb moisture and stays dry after the gel pack is removed. In trials, the gel pack remained cold out of the freezer for over 1 hour. Typically, medical professionals recommend icing for 20 minutes only and then reapplying ice after the area has warmed up to room temperature, therefore, the trials show the ice stays cold for much longer than necessary to permit adequate icing as suggested by a doctor. Ice should be used as often as possible during the first couple days of injury to aid recovery or post-operatively after a surgical procedure.

The design of sock 110 may provide the person wearing sock 110 (e.g., ICE-SOX) mobility while icing. For example, post-marathon or race, a runner may place these socks on and immediately start applying ice and compression to their calves and shins after crossing the finish line. Person 100 may also wear sock 110 to run a race Alternatively, person 100 can elevate their leg or foot while icing, or go to grab a beverage in the kitchen with the ice still on the top of their foot, for example.

Sock 110 (e.g., ICE-SOX) may provide comfort with a seamless design, and therefore, sock 110 caters to not only an injured audience, but also the athletic community. Sock 110 may also have a reinforced toe and heal for additional comfort. Person 100 may wear sock 110 (e.g., ICE-SOX) strictly as compression socks with sneakers during a workout or to run a race, and when done, have the ability to ice the area post workout or race, which is often important for athletes. Sock 110 may also have a 360 degree toe box construction and an anatomically accurate design for the left and right foot, providing more comfort to the wearer. Audiences see the emphasis on icing every time they watch pro football, basketball, hockey and baseball games, as footage always shows athletes on the sidelines icing.

While various embodiments of the present invention have been described above, it should be understood that they have been presented by way of example only, and not limitation. Thus, the breadth and scope of the present invention should not be limited by any of the above-described illustrative embodiments, but should instead be defined only in accordance with the following claims and their equivalents.

What is claimed is:

1. A compression wrap comprising:
a sock, the sock comprising:
a plurality of layers of fabric, the plurality of layers of fabric including an inner layer of fabric and an outer layer of fabric,
an upper rib band,
a middle rib band,
a pocket within the inner layer of fabric and the outer layer of fabric and between the upper rib band and the middle rib band, and
an opening in the outer layer of the plurality of layers of fabric, the opening providing access to the pocket, the pocket providing support for one or more cold or hot packs.

2. The compression wrap of claim 1, wherein the one or more cold or hot packs comprises one or more ice packs or gel packs.

3. The compression wrap of claim 1, wherein the one or more cold or hot packs provide icing or heating to a person wearing the sock.

4. The compression wrap of claim 3, wherein the icing or heating is provided to a calf and a shin of a leg of a person simultaneously.

5. The compression wrap of claim 1, wherein the sock provides compression to a person wearing the sock.

6. The compression wrap of claim 1, wherein the plurality of layers of fabric comprises nylon and spandex.

7. The compression wrap of claim 1, wherein the one or more cold or hot packs are inserted into the pocket through the opening.

8. The compression wrap of claim 1, wherein the plurality of layers of fabric are elastic and the one or more cold or hot packs are held in place by the elasticity of plurality of layers of fabric.

9. The compression wrap of claim 1, wherein the inner layer of fabric prevents the one or more cold or hot packs from directly contacting skin.

10. The compression wrap of claim 1, wherein the sock is capable of being pulled up to below a knee or folded down and pulled over a foot to form additional layers of fabric with compression and permitting icing or heating to a bottom of a foot, a top of the foot, a side of the foot, or an ankle.

11. A method for providing mobile compression and heat or icing, the method comprising:
wearing a compression sock comprising:
a plurality of layers of fabric, the plurality of layers of fabric including an inner layer of fabric and an outer layer of fabric,
an upper rib band,
a middle rib band,
a pocket within the inner layer of fabric and the outer layer of fabric and between the upper rib band and the middle rib band, and
an opening in the outer layer of the plurality of layers of fabric, the opening providing access to the pocket, the pocket providing support for one or more cold or hot packs; and
placing the one or more cold or hot packs into the pocket of the sock through the opening.

12. The method of claim 11, further comprising folding down a top portion of the sock including the upper rib band over a foot to provide an additional layer, with compression, to hold the one or more cold or hot packs on a top of a foot, a bottom of the foot, or a side of the foot, or an ankle.
